(19) United States
(12) Patent Application Publication
(10) Pub. No.: US 2011/0172428 A1
(43) Pub. Date: Jul. 14, 2011

(54) METHODS FOR THE PREPARATION OF INDAZOLE-3-CARBOXYLIC ACID AND N-(S)-1-AZABICYCLO[2.2.2]OCT-3-YL-1H-INDAZOLE-3-CARBOXAMIDE HYDROCHLORIDE SALT

(76) Inventors: Shan-Ming Kuang, Florence, SC (US); Pingsheng Zhang, Florence, SC (US)

(21) Appl. No.: 12/983,906

(22) Filed: Jan. 4, 2011

Related U.S. Application Data
(60) Provisional application No. 61/294,143, filed on Jan. 12, 2010.

(51) Int. Cl. C07D 453/02; C07D 231/56

(52) U.S. Cl. 546/133; 548/362.5

(57) ABSTRACT

The present invention provides novel methods for preparing indazole-3-carboxylic acid 2, a key starting material for the manufacture of agonists or partial agonists of the nicotinic α-7 receptor, such as N—(S)-1-azabicyclo[2.2.2]oct-3-yl-1H-indazole-3-carboxamide HCl salt 13. Nicotinic α-7 receptor agonists and partial agonists are being useful in the treatment of disease conditions associated with defective or malfunctioning nicotinic acetylcholine receptors, especially of the brain, such as for the treatment of Alzheimer's disease and schizophrenia, as well as other psychiatric and neurological disorders. The present methods are useful for preparing indazole-3-carboxylic acid on scaled-up levels.

### PRIORITY TO RELATED APPLICATIONS

This application claims the benefit of U.S. Provisional No. 61/294,143, filed Jan. 12, 2010, which is hereby incorporated by reference in its entirety.

### FIELD OF THE INVENTION

The present invention provides novel methods for preparing indazole-3-carboxylic acid 2, a key starting material for the manufacture of pharmaceuticals, such as N—(S)-1-azabicyclo[2.2.2]oct-3-yl-1H-indazole-3-carboxamide HCl salt 13, that are agonists or partial agonists of the nicotinic α-7 receptor. These actives are being studied for their use in the treatment of disease conditions associated with defective or malfunctioning nicotinic acetylcholine receptors, especially of the brain, such as for the treatment of Alzheimer's disease and schizophrenia, as well as other psychiatric and neurological disorders. The present methods are useful for the scaled-up preparation of compound 2.

### BACKGROUND OF THE INVENTION

Bicyclic indazole amides such as N—(S)-1-Azabicyclo[2.2.2]oct-3-yl-1H-indazole-3-carboxamide HCl salt 16 are useful for the treatment of Alzheimer's Disease and schizophrenia,

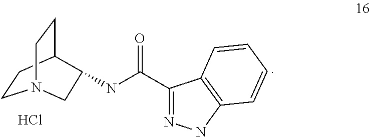

Bicyclic indazole amides are described in WO 2004/029050, WO 2005/063767, WO 2005/092890, WO 2005/111038, WO 2006/001894, WO 2006/069097, and WO 2007/038367. These compounds are nicotinic α-7 receptor partial agonists. Compounds that act on this receptor could be beneficial in the treatment of Alzheimer's disease and schizophrenia, as well as other psychiatric and neurological disorders.

Indazole-3-carboxylic acid 2 is a key starting material for the manufacture of compound 16, a nicotinic α-7 receptor partial agonist. To date, scale up reactions to prepare compound 2 safely and economically have not been successful.

There are two main methods for preparing indazole-3-carboxylic acid 2. One method is set out below in Scheme 1.

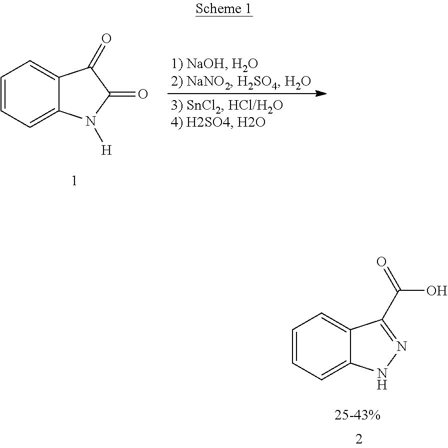

Isatin 1 is hydrolyzed with aqueous NaOH. The intermediate is then converted to a diazonium salt, followed by reduction to form an aryl hydrazine. Cyclization of the aryl hydrazine under acidic conditions affords the indazole acid (J. Am. Chem. Soc., 1952, 74, 2009; Faming Zhuanli Shenqing Gongkai Shuomingshu (2003), 11 pp. CODEN: CNXXEV CN 1451660 A 20031029 CAN 142:430447 AN 2005:203871 CAPLUS; Guangzhou Huagong (2000), 28(4), 108, 98. CODEN: GUHUEZ ISSN:1001-9677. CAN 135:19588 AN 2001:109038 CAPLUS). The low overall yield (25-43%) and the safety risks associated with the explosive nature of the diazonium salt intermediate make this method unsuitable for scale-up.

The second method is set out below in Scheme 2, (J. Heterocyclic Chem., 1989, 26, 531; Faming Zhuanli Shenqing Gongkai Shuomingshu (2008), 35 pp. CODEN: CNXXEV CN 101239950 A 20080813 CAN 149:332325 AN 2008:997937 CAPLUS; Yaoxue Jinzhan (2006), 30(5), 235-237. CODEN: YJAIBE ISSN:1001-5094. CAN 146:316842 AN 2006:770369 CAPLUS; Faming Zhuanli Shenqing Gongkai Shuomingshu (2005), 7 pp. CODEN: CNXXEV CN 1594297 A 20050316 CAN 144:192261 AN 2006:135971 CAPLUS.

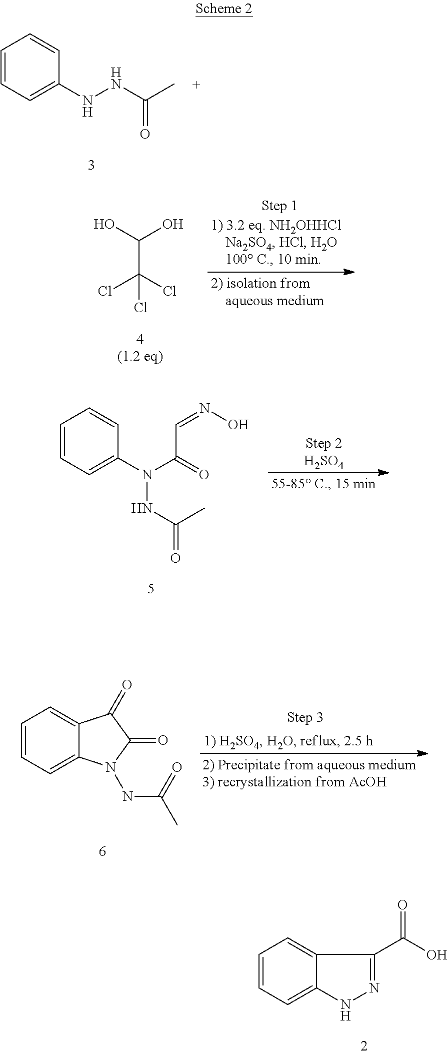

In step 1, compounds 3 and 4 are reacted to form compound 5. In step 2, compound 5 is treated with concentrated sulfuric acid to provide compound 6. The reaction mixture is diluted with water and heated to reflux to provide compound 2. This method contains three isolations with three chemical transformations and possesses some intrinsic shortcomings that affect its use on scale-up. These shortcomings include low capacity in step 1 and the use of 95% sulfuric acid as a solvent in step 2. Another shortcoming is the need to quickly add an aqueous solution of chloral hydrate 3 to an acidic reaction mixture at 100° C. in step 1, followed by a quick cooling of the mixture to ambient temperature in 10 minutes to avoid yield loss, an operation hard to achieve on large scale.

Accordingly, novel, efficient methods for preparing compound 2 on a scale up level are desirable.

### SUMMARY OF THE INVENTION

The present invention, as illustrated in Scheme 3, provides a method for preparing compound 2 having the formula:

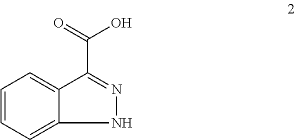

which comprises:
(a) reacting phenylhydrazine with benzaldehyde to provide benzaldehyde phenylhydrazone 9;

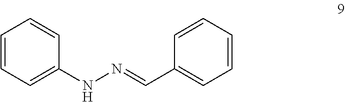

(b) admixing the benzaldehyde phenylhydrazone from step (a) with oxalyl chloride to provide intermediate 10;

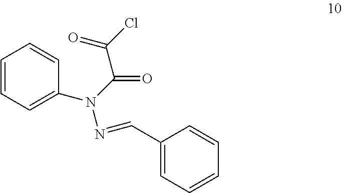

(c) admixing intermediate 10 from step (b) with aluminum chloride to provide intermediate 11; and

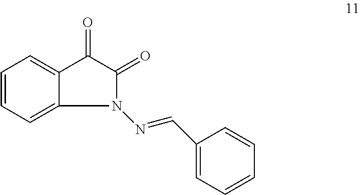

(d) admixing intermediate 11 from step (c) with an aqueous acidic solution to provide compound 2.

The present invention further provides a method for preparing compound 15, a free base form of 16, having the formula:

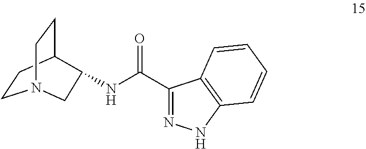

which comprises, as depicted in Scheme 4:
(a) admixing compound 2 with compound 12

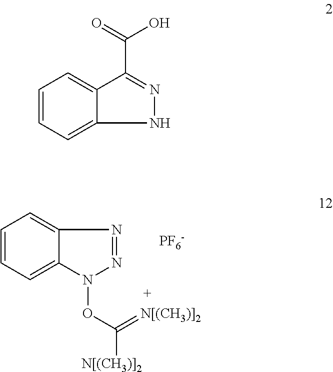

in the presence of a non-nucleophilic base and an inert organic solvent to provide intermediate 13; and

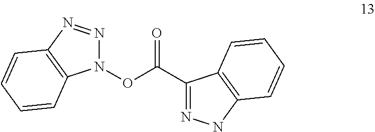

(b) adding compound 14,

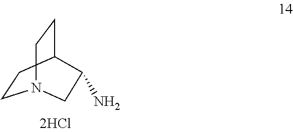

to intermediate 13 in step (a) to provide compound 15.

### BRIEF DESCRIPTION OF THE DRAWINGS

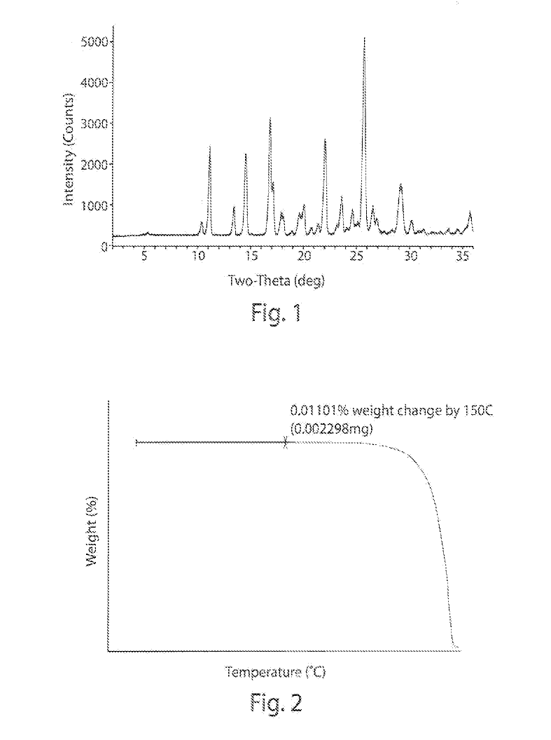

Form A of indazole-3-carboxylic acid can be characterized by at least three peaks selected from the following X-ray diffraction peaks obtained with a Cu_{Kα} radiation at 2θ (2 Theta)=10.3, 11.1, 13.3, 14.5, 16.8, 20.0, 22.0, 23.6, 25.7, and 29.2 (±0.2°) shown on FIG. 1.

Form A of indazole-3-carboxylic acid is a solvent-free form as no significant weight loss is observed in the TGA curve prior to decomposition as shown on FIG. 2.

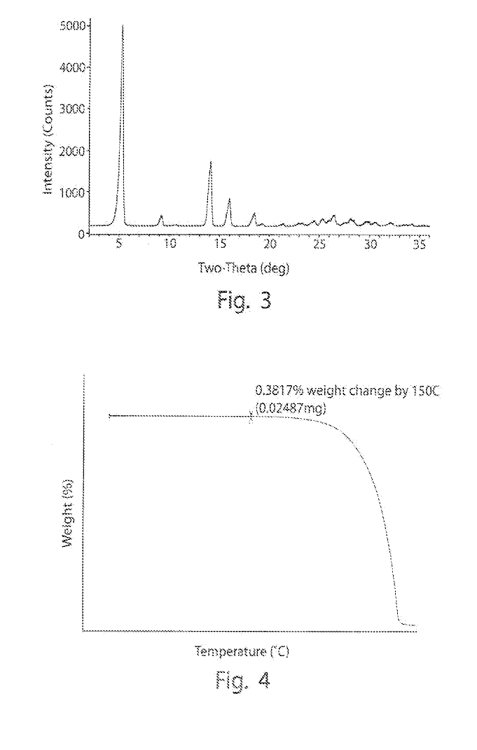

Form B of indazole-3-carboxylic acid is a solvent-free, crystalline form. Form B can be characterized by at least three peaks selected from the following X-ray diffraction peaks obtained with a Cu_{Kα} radiation at 2θ (2 Theta)=5.3, 9.2, 14.1, 16.0, 18.5, 19.3, 21.4, 23.3, 24.6, and 26.6 (±0.2°) shown on FIG. 3.

Form B of indazole-3-carboxylic acid is a solvent-free form as no significant weight loss is observed in the TGA curve prior to decomposition as shown on FIG. 4.

### DETAILED DESCRIPTION OF THE INVENTION

As used herein, the following terms have the meanings set out below.

The term “inert organic solvent” refers to an organic solvent that does not interfere chemically with the reaction. Non-limiting illustrative examples of inert organic solvents include dichloromethane, chloroform, dimethylformamide (DMF), and the like.

The term “non-nucleophilic base” refers to an organic base that is a very strong base but is a poor nucleophile. Non-limiting illustrative examples of non-nucleophilic bases include triethylamine (TEA), N,N-diisopropylethylamine (DIPEA), 1,8-diazabicyloundec-7-ene (DBU), and the like.

The term “aqueous acidic solution” refers to solutions in which the solvent is water and the pH level is less than 7.0. The word “aqueous” means dissolved in water.

An acid is any chemical compound that, when dissolved in water, gives a solution with a hydrogen ion activity greater than in pure water, i.e. a pH less than 7.0. Common examples of aqueous acidic solutions include acetic acid, hydrochloric acid (HCl), sulfuric acid, mixtures thereof, and the like, in water.

The term “free base form” refers to the pure basic form, generally of an amine, as opposed to its salt form. The amine may be an alkaloid and the free base form is commonly used to describe the unprotonated amine form of a compound. Many free base forms are unstable in their pure form and are often stored as salts. The salts usually exhibit greater water solubility. Common counterions include chloride, bromide, acetate and oxalate.

As set out above, the present invention provides a method for preparing compound 2 having the formula:

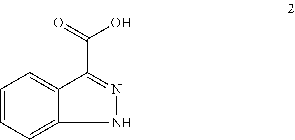

which comprises:
(a) reacting phenylhydrazine with benzaldehyde to provide benzaldehyde phenylhydrazone 9;

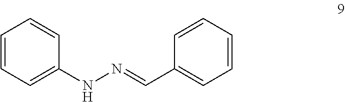

(b) admixing the benzaldehyde phenylhydrazone from step (a) with oxalyl chloride to provide intermediate 10;

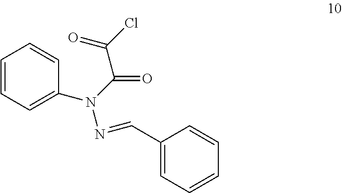

(c) admixing intermediate 10 from step (b) with aluminum chloride to provide intermediate 11; and

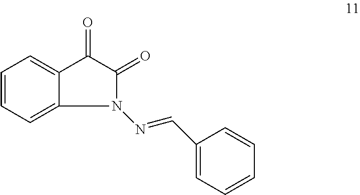

(d) admixing intermediate 11 from step (c) with an aqueous acidic solution to provide compound 2.

The method of the present invention for preparing compound 2 is set out below in Scheme 3.

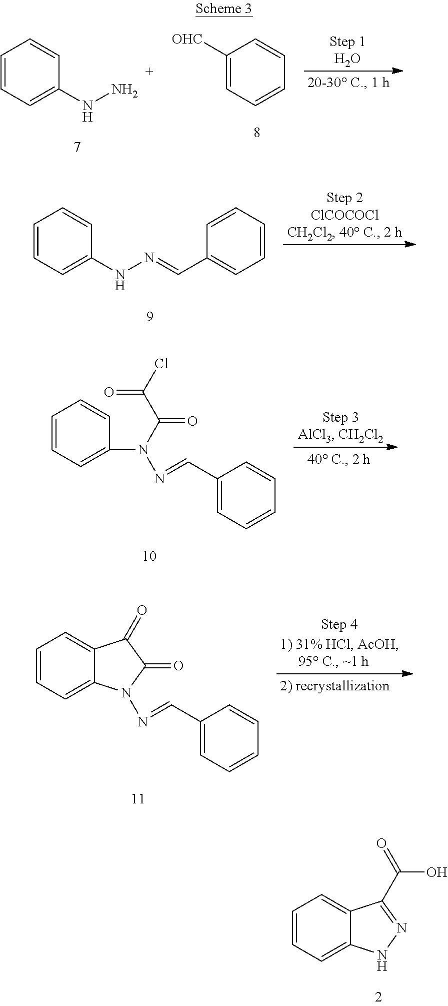

The present invention provides novel methods for preparing key intermediate indazole-3-carboxylic acid 2 via a diazonium free route that is safe and easily scalable. The novel method provides acid 2 in three steps starting from commercially available phenylhydrazine 7 and benzaldehyde 8. Reaction of phenylhydrazine 7 and benzaldehyde 8 yields benzaldehyde phenylhydrazone 9. Reaction of compound 9 with oxalyl chloride provides an intermediate 10, which is then treated with AlCl₃ in a Friedal-Crafts reaction to provide benzylideneaminoisatin 11. Hydrolysis and ring rearrangement of 11 produces the desired acid 2.

Preferably, step 1 is carried out in an aqueous medium or a mixed solvent of water and an alcohol (e.g. MeOH, EtOH, and 2-propanol) and is carried out at about 20-30° C., preferably 25-30° C., over about 1 h. Preferably, step 2 is carried out in an inert organic solvent, more preferably the organic solvent is dichloromethane, and step 2 is carried out at about 40° C., over about 2 h. Preferably, step 3 is carried out in an inert organic solvent, more preferably the organic solvent is dichloromethane, and is heated to reflux. Preferably, the aqueous acidic solution in step 4 is an aqueous mixture of acetic acid and hydrochloric acid and the aqueous acidic solution is mixed at about 90±5° C., over about 1 h.

Intermediate 11 can be isolated as a dichloromethane solution via extractive workup. Upon the completion of step 3 reaction, the reaction mixture is quenched by adding water. Dichloromethane solution of 11 is obtained through layer separation. This solution of 11 is then used in step 4 (Example 2, A). In an alternative process, 11 can be isolated as a wet cake solid via precipitation. In this case, step 3 reaction is quenched by adding water. Then, organic solvent is removed from the mixture by distillation. Intermediate 11 precipitates from the aqueous mixture and is isolated via filtration. The solid wet cake 11 is then used in step 4 (Example 2, B).

Indazole-3-carboxylic acid (2) exists in at least two different crystal forms (Form A and Form B). The crystal forms are identified using XRPD and TGA. “XRPD” is used herein as an acronym of X-Ray Powder Diffraction. X-ray diffraction patterns were recorded at ambient conditions with a Bruker D8 Advance powder X-ray diffractometer equipped with a CuKα radiation, a rotation sample stage, and a Vantec position sensitive detector. The samples were scanned from 2 to 36° 2θ at a step size of 0.007° and a step time of 0.35 s.

“TGA” is used herein as an acronym of ThermoGravimetric Analysis. TGA curves were measured on a TGA Q5000 from TA Instruments. System suitability tests and calibrations were carried out according to the internal standard operation procedure. The heating rate was 10° C./min with a nitrogen purge maintained throughout the run.

Indazole-3-carboxylic acid can be isolated, depending upon the method of preparation, as different polymorphs. Form A of indazole-3-carboxylic acid can be isolated from DMF/water, DMF/acidic water, or acetic acid. Form B of indazole-3-carboxylic acid can be isolated from dichloromethane, t-butyl methyl ether (MTBE), or ethyl acetate, etc. Form A of indazole-3-carboxylic acid can be obtained by suspending Form B of indazole-3-carboxylic acid in refluxing MeOH for 4 h. Form A of indazole-3-carboxylic acid is a solvent-free, crystalline form.

As set out above, the present invention further provides a method for preparing compound 15:

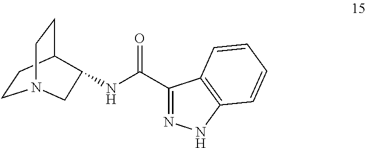

which comprises:
(a) admixing compound 2 with compound 12

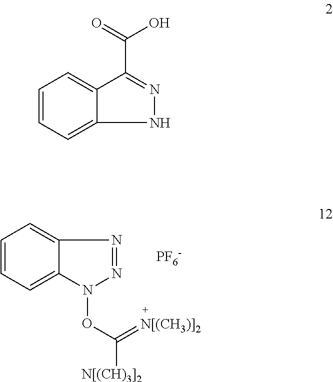

in the presence of a non-nucleophilic base and an inert organic solvent to provide intermediate 13; and

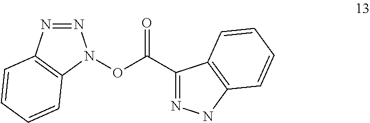

(b) adding compound 14,

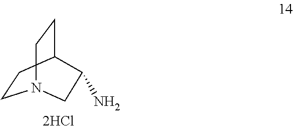

to intermediate 13 in step (a) to provide compound 15.

The method of the present invention for preparing N—(S)-1-azabicyclo[2.2.2]oct-3-yl-1H-indazole-3-carboxamide 15 from indazole-3-carboxylic acid 2, is set out below in Scheme 4.

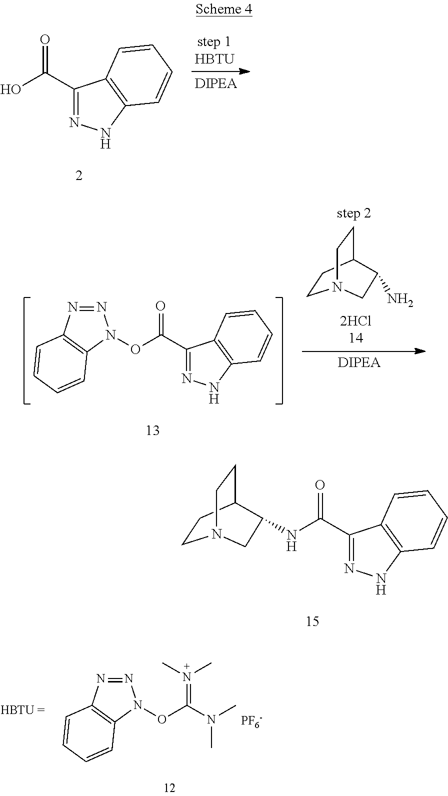

The non-nucleophilic base in step 1 is preferably diisopropylethylamine. The inert organic solvent in step 1 is preferably dimethylformamide. After addition of compound 14 to intermediate 13 in step 2, the reaction mixture is preferably stirred at room temperature overnight, then heated at 45° C. for 10 h. Compound 15 produced in step 2 is isolated, preferably by removing the inert organic solvent. Much lower cost and easy handling are benefits of this method.

The compounds of the present invention can be prepared according to the examples set out below. The examples are presented for purposes of demonstrating, but not limiting, the preparation of the compounds and compositions of this invention.

### EXAMPLES

### Example 1

### Preparation of Benzaldehyde phenylhydrazone 9 (Step 1)

### A. Using Pure Water as a Reaction Solvent

A 5 L, half jacketed, 4-necked round bottom flask equipped with a mechanic stirrer, a thermocouple, a nitrogen inlet, and an addition funnel was charged with 322 g of phenylhydrazine (2.98 Mol) and 3.3 L of water. Agitation was started. To the mixture was slowly charged 301 g of benzaldehyde (2.84 Mol) in ˜1 h, while maintaining batch temperature at 25-30° C. After the addition the mixture was stirred at 25-30° C. for at least 2 h and then cooled to 20° C. The solid was filtered and washed with 444 g of isopropyl alcohol (IPA). The wet cake was dried under vacuum at 70° C. overnight to give 540.4 g (97%) of benzaldehyde phenylhydrazone 9.

### B. Using Mixed Solvent of Water/2-Propanol as Reaction Solvent

A 3 L, half jacketed, 4-necked round bottom flask equipped with a mechanical stirrer, a thermocouple, a nitrogen inlet, and an addition funnel, was charged with 200 g of phenylhydrazine (1.85), 2.0 L of water, and 0.6 L of 2-propanol. Agitation was started. To the mixture was slowly charged 188 g of benzaldehyde (1.77 Mol) in ˜1 h, while maintaining batch temperature at 25-30° C. After the addition, the mixture was stirred at 25-30° C. for at least 2 h and then cooled to 20° C. The solid was filtered and washed with 2×250 mL of water/2-propanol (3:1, v/v). The wet cake was dried under vacuum at 90° C. for 20 h to give 335 g (96.4%) of benzaldehyde phenylhydrazone 9.

### Example 2

### Preparation of Indazole-3-carboxylic acid (2) from 9 (Steps 2 to 4)

### A. No Isolation of Intermediate 11

A 3 L, half jacketed, 4-necked round bottom flask equipped with a mechanical stirrer, a thermocouple, a nitrogen inlet, and an addition funnel, was charged with 35.6 g of oxalyl chloride (280 mMol) and 200 mL of dichloromethane. The solution was heated to ˜40° C., and a solution of 50 g of benzaldehyde phenylhydrazone 9 (255 mMol) in 800 mL of dichloromethane was added slowly. After the addition, the mixture was stirred at ˜40° C. for at least 2 h to generate intermediate solution I.

A second 3 L, half-jacketed, 4-necked round bottom flask equipped with a mechanical stirrer, a thermocouple, a nitrogen inlet, and an additional funnel was charged with 81.4 g of aluminum chloride (612 mMol) and 200 mL of dichloromethane. Agitation was started, and the intermediate solution I in the first flask was slowly charged while maintaining batch temperature below 30° C. After the addition the mixture was refluxed for at least 2 h. The mixture was cooled to ˜0° C., and 500 mL of water was slowly added while maintaining batch temperature <10° C. After the addition the mixture was agitated for ˜0.5 h. The agitation was stopped for layer separation. The bottom organic layer was separated. To the batch was added 100 mL of dichloromethane. The mixture was agitated for ˜0.5 h. The agitation was stopped for layer separation. The bottom organic layer was separated. The combined organic solution was washed with 400 mL of 10% HCl, followed by 400 mL of brine. The organic solution was concentrated to dryness to give 60.1 g intermediate 11 (95%).

A 500 mL, half jacketed, 4-necked round bottom flask equipped with a mechanic stirrer, a thermocouple, and a nitrogen inlet was charged with 28.7 g of intermediate 11 (115 mMol), 215 ml, of acetic acid, 43 mL of water and 28.7 g of 31% HCl. The mixture was agitated at 90±5° C. for 1 h and then was concentrated to dryness. To the flask was added 300 ml, of acetic acid. The mixture was agitated at 115° C. for ˜0.5 h, cooled to ambient temperature under agitation, and was aged for at least 1 h. The solid was filtered, and the wet cake was washed with 50 mL of acetic acid and dried under vacuum at 65° C. overnight to give 13.2 g (71% yield) of 2, as a mixture of Form A and B.

### B. With Isolation of 11 as Wet Cake

A 3 L, half jacketed, 4-necked round bottom flask equipped with a mechanic stirrer, a thermocouple, a nitrogen inlet, and an addition funnel, was charged with 71.2 g of oxalyl chloride (560 mMol) and 400 mL of dichloromethane. The solution was heated to ˜40° C. and a solution of 100 g of benzaldehyde phenylhydrazone (9) (509 mMol) in 1600 mL of dichloromethane was added slowly in ˜2 h. After the addition the mixture was stirred at ˜40° C. for at least 1 h to complete the reaction. Atmospheric distillation was started to remove ˜500 mL liquid and the batch was then cooled to 20° C. to afford intermediate solution I.

A second 3 L, half jacketed, 4-necked flask equipped with a mechanic stirrer, a thermocouple, a nitrogen inlet, and an addition funnel, was charged with 162 g of aluminum chloride (1220 mMol) and 300 mL of dichloromethane. Agitation was started and the intermediate solution I in the first flask was slowly charged while maintaining batch temperature below 30° C. After the addition the mixture was reflux for at least 2 h. Atmospheric distillation was started to remove ˜1000 mL of dichloromethane. The mixture was then cooled to ˜0° C. and 1000 mL of water was slowly added while maintaining batch temperature <20° C. After the addition the mixture was agitated for ˜0.5 h and was heated to distill out the rest of dichloromethane. To the batch was added 400 mL of water and the mixture was stirred at the temperature for ˜0.5 h. The resulting slurry was cooled to ˜20° C. and aged for at least 1 h. The solid was filtered, washed with 300 mL of water to give crude wet cake 9.

A 3 L, half jacketed, 4-necked round bottom flask equipped with a mechanic stirrer, a thermocouple, and a nitrogen inlet, was charged with the wet cake 9, followed by the addition of 900 mL of acetic acid, and 132 g of 31% HCl solution. The mixture was agitated, heated at 90±5° C. for 1 h and then was concentrated to the minimum stirring volume. To the flask was added 150 mL of N,N-dimethylacetamide (DMA). The distillation was continued until a total of 1040 mL of distillate was collected. The mixture was then cooled to ˜40° C. and 1000 mL of dichloromethane was slowly added. After the addition the mixture was refluxed for at least ˜1 h, cooled to 15° C. and aged for at least 1 h. The solid was filtered, the wet cake was washed with 300 mL of dichloromethane, dried under vacuum at 90° C. overnight to give 63 g (76% yield) of indazole-3-carboxylic acid (2), as pure form B.

### Example 3

### Conversion of Form B to Form A

A 1 L, half jacketed, 4-necked round bottom flask equipped with a mechanic stirrer, a thermocouple, and a condenser, was charged with 100 g of indazole-3-carboxylic acid (Form B) (0.62 Mol) and 200 mL of N,N-dimethylformamide (DMF). Agitation was started and the mixture was heated to ˜80° C. The mixture was stirred for 0.5 h to become a clear solution. To this solution was slowly added 600 mL of 5% hydrochloric acid in 2 h, resulting in precipitation. After addition of the hydrochloric acid, the mixture was stirred for 0.5 h, maintaining the ˜80° C. temperature. The mixture then was cooled to ˜10° C. in approximately 2 h. The mixture was aged for at least 1 h. The solid was filtered, washed with 200 mL of water, and dried under vacuum at 90° C. for 15 h to give 95.1 g (95.1%) of indazole-3-carboxylic acid (Form A)

### Example 4

### Preparation of N—(S)-1-Azabicyclo[2.2.2]oct-3-yl-1H-indazole-3-carboxamide

To a mixture of 2.5 kg indazole-3-carboxylic acid 2 and 13.4 L N,N-diisopropylethylamine in 23 L of dimethylformamide (DMF) was added 5.84 kg of O-benzotrizole-N,N,N′,N′-tetramethyl-uronium-hexafluoro-phosphate (HBTU). The resulting mixture was stirred at room temperature for 2 h to provide intermediate 13. Then, 2.56 kg of (S)-3-aminoquinuclidine dihydrochloride 14 was added to intermediate 13. The reaction mixture was stirred at room temperature overnight, then heated at 45° C. for 10 h. The mixture was then concentrated to remove most of the DMF. To the residue was added 80 L of dichloromethane. The mixture was stirred for 3 h. The solid was filtered, washed with 20 L dichloromethane, and dried under vacuum to yield 3.07 kg of compound 15.

While a number of embodiments of this invention have been represented, it is apparent that the basic construction can be altered to provide other embodiments that utilize the invention without departing from the spirit and scope of the invention. All such modifications and variations are intended to be included within the scope of the invention as defined in the appended claims rather than the specific embodiments that have been presented by way of example.

We claim:

1. A method for preparing compound 2: 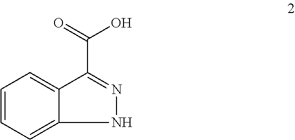
which comprises:
(a) reacting phenylhydrazine with benzaldehyde to provide benzaldehyde phenylhydrazone 9; 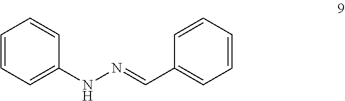
(b) admixing the benzaldehyde phenylhydrazone from step (a) with oxalyl chloride to provide intermediate 10; 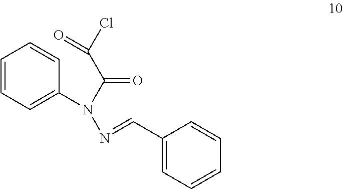
(c) admixing intermediate 10 from step (b) with aluminum chloride to provide intermediate 11; and 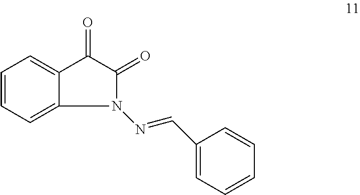
(d) admixing intermediate 11 from step (c) with an aqueous acidic solution to provide compound 2.

2. The method according to claim 1, wherein step (a) is carried out in an aqueous medium.

3. The method according to claim 1, wherein step (a) is carried out at about 25-30° C.

4. The method according to claim 1, wherein step (b) is carried out in an inert organic solvent.

5. The method according to claim 4, wherein the organic solvent is dichloromethane.

6. The method according to claim 1, wherein step (b) is carried out at about 40° C.

7. The method according to claim 1, wherein step (c) is carried out in an inert organic solvent.

8. The method according to claim 7, wherein the organic solvent is dichloromethane.

9. The method according to claim 1, wherein after the admixing in step (c), the admixture is heated to reflux.

10. The method according to claim 1, wherein the aqueous acidic solution in step (d) is an aqueous mixture of acetic acid and hydrochloric acid.

11. The method according to claim 1, wherein the aqueous acidic solution in step (d) is mixed at about 90±5° C.

12. A method for preparing compound 15: 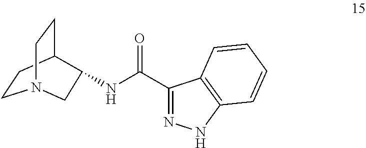
which comprises:
(a) admixing compound 2 with compound 12 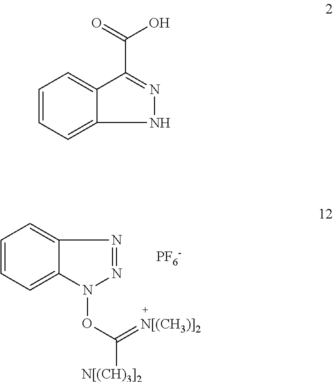
in the presence of a non-nucleophilic base and an inert organic solvent to provide intermediate 13; and 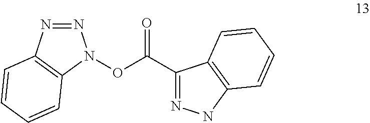
(b) adding compound 14, 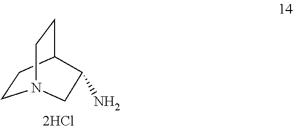
to intermediate 13 in step (a) to provide compound 15.

13. The method according to claim 12, wherein the non-nucleophilic base in step (a) is diisopropylethylamine.

14. The method according to claim 12, wherein the inert organic solvent in step (a) is dimethylformamide.

15. The method according to claim 12, wherein compound 15 in step (b) is isolated by removing the inert organic solvent.

16. A crystalline form A of indazole-3-carboxylic acid 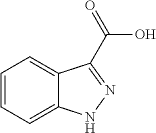
having at least three peaks selected from the following X-ray diffraction peaks obtained with CU_{kα} radiation, expressed in degrees 2θ=10.3, 11.1, 13.3, 14.5, 16.8, 20.0, 22.0, 22.0, 23.6, 25.7, and 29.2.

17. A crystalline form B of indazole-3-carboxylic acid 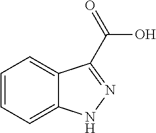
having at least three peaks selected from the following X-ray diffraction peaks obtained with CU_{kα} radiation, expressed in degrees 2θ=5.3, 9.2, 14.1, 16.0, 18.5, 19.3, 21.4, 23.3, 24.6, and 26.6.

18. A method for preparing crystalline form A of indazole-3-carboxylic acid which comprising
a) dissolving crystalline form B of indazole-3-carboxylic acid in N,N-dimethylformamide;
b) adding 5% hydrochloric acid over a period of about 2 hours;
c) stirring the mixture for approximately 30 minutes, followed by cooling to about 10° C. over a two hour periods;
d) aging the mixture for at least 1 hour; and
e) filtering the resulting solid, followed by washing with water and vacuum drying.
